(19) United States
(12) Patent Application Publication
(10) Pub. No.: US 2026/0021081 A1
(43) Pub. Date: Jan. 22, 2026

(54) INHALATION DEVICE FOR 7-OH-MITRAGYNINE

(71) Applicant: Sunflower Wellness, Inc., Hudson, CO (US)

(72) Inventors: Westley CRUCES, Hudson, CO (US); Giovanni RAMIREZ, Hudson, CO (US); Tesfay TESFATSION, Hudson, CO (US); Kyle RAY, Hudson, CO (US)

(21) Appl. No.: 18/794,857

(22) Filed: Aug. 5, 2024

Related U.S. Application Data
(60) Provisional application No. 63/674,057, filed on Jul. 22, 2024.

(51) Int. Cl. A61K 31/4375; A61K 9/00; A61K 31/437; A61K 45/06; A61P 29/00

(52) U.S. Cl. CPC A61K 31/4375; A61K 9/007; A61K 31/437; A61K 45/06; A61P 29/00

(57) ABSTRACT

Compositions comprising at least 10% of a compound of formula (I) by weight, at least 10% of a compound of formula (II) by weight, or at least 10% by weight of a combination of a compound of formula (I) and a compound of formula (II), are disclosed herein. Methods for administering the compositions are disclosed herein. The methods can be used to produce a calming effect, produce an analgesic effect, manage pain, treat cough, treat anxiety, treat depression, treat opioid use disorder, and/or to treat opioid withdrawal in a subject.

This application claims priority from U.S. Provisional Application No. 63/674,057, filed Jul. 22, 2024, which is hereby incorporated by reference in its entirety.

### BACKGROUND OF THE INVENTION

### I. Field of the Invention

This invention relates to the field of organic chemistry.

### II. Background

Mitragyna speciosa, also known as kratom, is an herbal leaf that is native to southeast Asia.¹⁻³ Kratom has traditionally been used for its opioid- and stimulant-like medicinal properties, and is consumed via chewing, smoking, and ingesting tea made from its leaves. Chemical constituents of kratom leaf include greater than 50 alkaloid natural products, with mitragynine, 7-hydroxymitragynine, raubasine, speciociliatine, paynantheine, corynantheidine, speciogynine, mitraphylline, rhynchophylline, and mitralactonal representing the major alkaloid constituents. Mitragynine and 7-hydroxymitragynine are primarily responsible for the medicinal effects of kratom, with the other alkaloids thought to create a synergistic effect.^{5,6} Both mitragynine and 7-hydroxymitragynine are agonists of the mu opioid receptor, with 7-hydroxymitragynine having a ˜40-fold greater potency than mitragynine and a ˜10-fold greater potency than morphine.⁷

Administration of 7-hydroxymitragynine via inhalation methods is limited, as the primary route of administration is oral administration of capsuled powders or compounded pills. Inhalation-based 7-hydroxymitragynine delivery devices, exist, however, many of these include low-concentration 7-hydroxymitragynine solutions that provide 7-hydroxymitragynine in small amounts. There exists a need for 7-hydroxymitragynine administration methods that provide increased onset rates over oral formulations and provide high 7-hydroxymitragynine dosages, in comparison to existing delivery devices.

### SUMMARY OF THE INVENTION

The present inventors have developed a method for isolating and administering compounds of formula (I), compound of formula (II), and/or related compounds that improves on currently available methods. The methods involve the use of compositions that comprise high concentrations of a compound of formula (I) and/or a compound of formula (II), where R is H, halide, alkyl, aryl, nitrile or —CONH₂. By employing compositions comprising high concentrations of a compound of formula (I) and/or a compound of formula (II), the amount of compound contained within each administered dose is increased. This provides a higher total dose, as compared to currently-available methods. The methods of administering a compound of formula (I) and/or a compound of formula (II) disclosed herein can be used to produce a calming effect and/or an analgesic effect in a subject. The methods of administering a compound of Formula (I) and/or a compound of formula (II) disclosed herein can be used to manage pain in a subject, treat cough, treat anxiety, treat depression, treat opioid use disorder, and/or to treat opioid withdrawal in a subject. In some embodiments, a compound of formula (I) is 7-hydroxymitragynine. In some embodiments, a compound of formula (II) is mitragynine pseudoindoxyl.

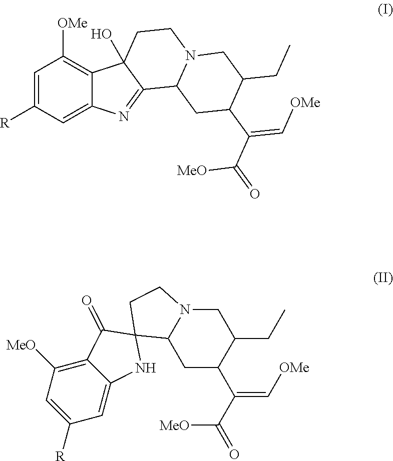

Some embodiments of the present disclosure are directed to compositions comprising methods of administering a compound of formula (I) and/or a compound of formula (II). In some embodiments, the compositions disclosed herein comprise at least 10% of a compound of formula (I) by weight, based on the total weight of the composition. In some embodiments, a composition as disclosed herein contains any one of, less than, greater than, between, or any range thereof of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% a compound of formula (I) by weight, based on the total weight of the composition. In some embodiments, a composition as disclosed herein contains any one of, less than, greater than, between, or any range thereof of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% a compound of formula (II) by weight, based on the total weight of the composition. In some embodiments, the composition comprising a compound of formula (I) and/or a compound of formula (II) further comprises a solvent. In some embodiments, the solvent is provided in an amount ranging from 1% to 90% by weight. In some embodiments, a composition as disclosed herein contains any one of, less than, greater than, between, or any range thereof of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of a solvent by weight, based on the total weight of the composition. In some embodiments, the compositions disclosed herein are in liquid form. Non-limiting examples of liquid compositions include oils, solutions in which the a compound of formula (I) and/or a compound of formula (II) are dissolved, and suspensions in which the a compound of formula (I) and/or a compound of formula (II) are suspended. In some embodiments, the compositions disclosed herein can be included in an ingestible product. Non-limiting examples of ingestible products include tablets, capsules, tablet, capsule, lozenges, candy, gummy, chocolate, brownie, cookie, trail bar, cracker, dissolving strip, and mint.

Some embodiments of the present disclosure are directed to a method for administering a composition comprising a compound of formula (I) and/or a compound of formula (II) to a subject. In some embodiments, the method comprises providing a device comprising a composition comprising at least 10% a compound of formula (I), at least 10% a compound of formula (II), or at least 10% of a combination of a compound of formula (I) and a compound of formula (II) by weight, based on a total weight of the composition, volatilizing the composition within the device, and inhaling the volatilized composition, wherein inhaling the composition from the device effects administration of the composition to the subject. In some embodiments, the composition comprises a solvent. In some embodiments, the solvent is provided in an amount ranging from 1% to 90% by weight, based on a total weight of the composition. In some embodiments, the composition further comprises at least one of an antioxidant, a preservative, a flavoring agent, a surfactant, and a tobacco material. In some embodiments, the device is a vaporizing device or an aerosolizing device. In some embodiments, the compositions of the present disclosure can be administered orally, via inhalation (e.g., aerosol inhalation), intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, locally, via injection, via infusion, via continuous infusion, via localized perfusion bathing target cells directly, via a catheter, via a lavage, in cremes, or in lipid compositions (e.g., liposomes).

Some embodiments of the present disclosure are directed to a method for inducing an analgesic effect in a subject. In some embodiments, the method comprises providing a device comprising a composition comprising at least 10% a compound of formula (I), at least 10% a compound of formula (II), or at least 10% of a combination of a compound of formula (I) and a compound of formula (II) by weight, based on a total weight of the composition, volatilizing the composition within the device, and inhaling the volatilized composition, wherein inhalation of the composition from the device leads to an analgesic effect in the subject. In some embodiments, the composition comprises a solvent. In some embodiments, the solvent is provided in an amount ranging from 1% to 90% by weight, based on a total weight of the composition. In some embodiments, the composition further comprises at least one of an antioxidant, a preservative, a flavoring agent, and a tobacco material. In some embodiments, the device is a vaporizing device or an aerosolizing device. In some embodiments, an analgesic effect can be induced in a subject by administering a composition of the present disclosure orally, via inhalation (e.g., aerosol inhalation), intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, locally, via injection, via infusion, via continuous infusion, via localized perfusion bathing target cells directly, via a catheter, via a lavage, in cremes, or in lipid compositions (e.g., liposomes).

Some embodiments of the present disclosure are directed to a method for inducing an analgesic effect in a subject through administration of a composition. In some embodiments, the method comprises administering to the subject a composition comprising at least 10% a compound of formula (I), at least 10% a compound of formula (II), or at least 10% of a combination of a compound of formula (I) and a compound of formula (II) by weight, based on a total weight of the composition, wherein administration of the composition leads to an analgesic effect in the subject. In some embodiments, the composition is a liquid. In some embodiments, the liquid composition comprises a solvent. In some embodiments, the solvent is provided in an amount ranging from 1% to 90% by weight, based on a total weight of the liquid composition. In some embodiments, the liquid composition further comprises at least one of an antioxidant, a preservative, a flavoring agent, and a tobacco material. In some embodiments, the administration is oral.

In some embodiments, the methods disclosed herein are used to administer compositions comprising a compound of formula (I), a compound of formula (II), or a combination of a compound of formula (I) and a compound of formula (II). In some embodiments, the methods disclosed herein are used to administer compositions comprising a compound of formula (I) and/or a compound of formula (II) through inhalation. In other embodiments, the methods disclosed herein are used to orally administer liquid compositions comprising a compound of formula (I) and a compound of formula (II). In some embodiments, the methods disclosed herein are used to administer vaporized compositions comprising a compound of formula (I) and a compound of formula (II), where the vaporized compositions are obtained by vaporizing liquid compositions comprising a compound of formula (I) and a compound of formula (II). In some embodiments, the methods disclosed herein are used to administer aerosolized compositions comprising a compound of formula (I) and a compound of formula (II), where the aerosolized compositions are obtained by employing a propellant gas to aerosolize a liquid or vaporized composition comprising a compound of formula (I) and a compound of formula (II). In some embodiments, the methods disclosed herein are used to manage pain in a subject, treat cough, treat anxiety, treat depression, treat opioid use disorder, and/or to treat opioid withdrawal in a subject, wherein the methods comprise administering a composition as disclosed herein.

Some embodiments of the present disclosure are directed to a device that comprises a composition that includes a high-concentration a compound of formula (I) and a compound of formula (II). In some embodiments, the composition within the vaporizing device comprises at least 10% a compound of formula (I), at least 10% a compound of formula (II), or at least 10% of a combination of a compound of formula (I) and a compound of formula (II), by weight, based on a total weight of the composition. In some embodiments, the device is a vaporizing device or an aerosolizing device.

In the context of the present invention, at least the following 30 aspects are described. Aspect 1 includes a composition comprising at least 10% a compound of formula (I) by weight, at least 10% of a compound of formula (II) by weight, or at least 10% by weight of a combination of a compound of formula (I) and a compound of formula (II), based on a total weight of the composition, where R is H, halide, alkyl, aryl, nitrile or —CONH₂. Aspect 2 depends on Aspect 1, wherein the composition comprises from 10% to 99% by weight of a compound of formula (I), based on a total weight of the composition. Aspect 3 depends on Aspect 1, wherein the composition comprises from 10% to 99% by weight of the compound of formula (II), based on a total weight of the composition. Aspect 4 depends on Aspect 1, wherein the composition comprises a solvent in an amount ranging from 1% to 90% by weight, based on a total weight of the composition. Aspect 5 depends on Aspect 1, wherein the compound of formula (I) is 7-hydroxymitragynine. Aspect 6 depends on Aspect 1, wherein the compound of formula (II) is mitragynine pseudoindoxyl. Aspect 7 depends on Aspect 1, wherein the composition further comprises at least one of an antioxidant, a preservative, a flavoring agent, a surfactant, and a tobacco material. Aspect 8 includes a method for administering a composition comprising a compound of formula (I) and/or a compound of formula (II) to a subject. The method comprises obtaining a composition comprising at least 10% a compound of formula (I), at least 10% of a compound of formula (II), or at least 10% of a combination of a compound of formula (I) and by weight, based on a total weight of the composition, and administering the composition to the subject. Aspect 9 depends on Aspect 8, wherein the composition comprises from 10% to 99% by weight of a compound of formula (I), based on the total weight of the composition. Aspect 10 depends on Aspect 8, wherein the composition comprises from 10% to 99% by weight of the compound of formula (II), based on the total weight of the composition. Aspect 11 depends on Aspect 8, wherein the composition is formulated as an oil, solution, suspension, tablet, capsule, lozenge, candy, gummy, chocolate, brownie, cookie, trail bar, cracker, dissolving strip, or mint. Aspect 12 depends on Aspect 8, wherein the composition is formulated as a liquid. Aspect 13 depends on Aspect 8 wherein the composition comprises a solvent in an amount ranging from 1% to 90% by weight, based on a total weight of the composition. Aspect 14 depends on Aspect 8, wherein the compound of formula (I) is 7-hydroxymitragynine. Aspect 15 depends on Aspect 8, wherein the compound of formula (II) is mitragynine pseudoindoxyl. Aspect 16 depends on Aspect 8, wherein the composition further comprises at least one of an antioxidant, a preservative, a flavoring agent, a surfactant, and a tobacco material. Aspect 17 depends on Aspect 8, wherein the method further comprises volatilizing the composition and inhaling the volatilized composition. Aspect 18 includes a method for inducing an analgesic effect in a subject. The method comprises obtaining a composition comprising at least 10% a compound of formula (I), at least 10% of a compound of formula (II), or at least 10% of a combination of a compound of formula (I) and a compound of formula (II) by weight, based on a total weight of the composition, and administering the composition to the subject, wherein administration of the composition leads to an analgesic effect in the subject. Aspect 19 depends on Aspect 18, wherein the composition comprises from 10% to 99% by weight of a compound of formula (I), based on the total weight of the composition. Aspect 20 depends on Aspect 18, wherein the composition comprises from 10% to 99% by weight of a compound of formula (II), based on the total weight of the composition. Aspect 21 depends on Aspect 18, wherein the composition is formulated as an oil, solution, suspension, tablet, capsule, lozenge, candy, gummy, chocolate, brownie, cookie, trail bar, cracker, dissolving strip, or mint. Aspect 22 depends on Aspect 18, wherein the composition is formulated as a liquid. Aspect 23 depends on Aspect 18, wherein the composition comprises a solvent in an amount ranging from 1% to 90% by weight, based on a total weight of the composition. Aspect 24 depends on Aspect 18, wherein the composition further comprises at least one of an antioxidant, a preservative, a flavoring agent, and a tobacco material. Aspect 25 depends on Aspect 18, wherein the method further comprises volatilizing the composition; and inhaling the volatilized composition. Aspect 26 depends on Aspect 18, wherein the compound of formula (I) is 7-hydroxymitragynine. Aspect 27 depends on Aspect 18,herein the compound of formula (II) is mitragynine pseudoindoxyl. Aspect 28 includes a device comprising a composition that includes at least 10% a compound of formula (I) by weight, at least 10% a compound of formula (II) by weight, or at least 10% by weight of a combination of a compound of formula (I) and a compound of formula (II), based on a total weight of the composition. Aspect 29 depends on Aspect 28, wherein the compound of formula (I) is 7-hydroxymitragynine. Aspect 30 depends on Aspect 28, wherein the compound of formula (II) is mitragynine pseudoindoxyl.

As used herein, the term “volatilize” means to cause a substance to evaporate or disperse in vapor. The term “volatile” refers to a chemical substance that that is readily vaporizable at relatively low temperature (relative to a nonvolatile substance). As used herein, the term “isolating” means to separate a desired natural from a raw material.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a photograph of a volcano hybrid vaporizer that was employed in various experiments disclosed herein.

FIG. 2 is a table that includes administration route and 7-hydroxymitragynine dosages for test mice used in tail flick studies.

FIG. 3 is a table that includes tail flick test results of oral administration of 7-hydroxymitragynine in wildbred mice.

FIG. 4 is a table that includes tail flick test results of inhalation administration of 7-hydroxymitragynine in wildbred mice.

FIGS. 5A-5D. FIG. 5A tail flick test results of oral administration of 7-hydroxymitragynine in wildbred mice at 25° C. FIG. 5B of tail flick test results of inhalation administration of 7-hydroxymitragynine in wildbred mice at 25° C. FIG. 5C of tail flick test results of oral administration of 7-hydroxymitragynine in wildbred mice at 30° C. FIG. 5D of tail flick test results of inhalation administration of 7-hydroxymitragynine in wildbred mice at 30° C. FIG. 5E of tail flick test results of oral administration of 7-hydroxymitragynine in wildbred mice at 40° C.

FIG. 5F of tail flick test results of inhalation administration of 7-hydroxymitragynine in wildbred mice at 40° C.

FIG. 6 is a table that includes 7-hydroxymitragynine doses administered to mice, and the equivalent translated dose for an adult human.

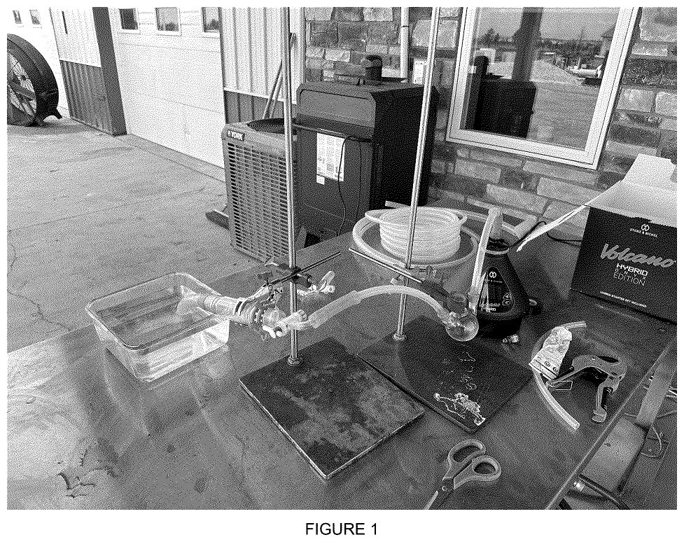

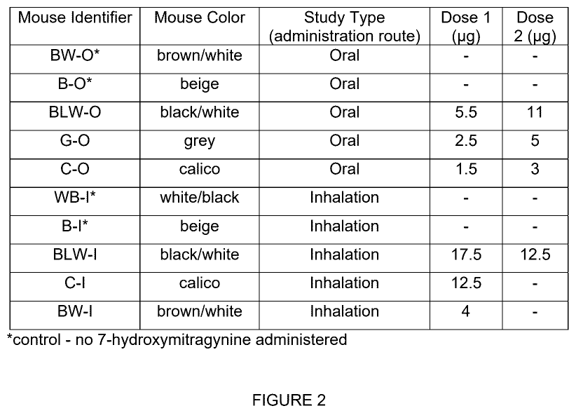

### DETAILED DESCRIPTION OF THE INVENTION

The present inventors have discovered methods for increasing the dose of compounds of formula (I), e.g., 7-hydroxymitragynine and compound of formula (II), e.g., mitragynine pseudoindoxyl, delivered to a subject. The methods involve administration of compositions that comprise a high concentration of a compound of formula (I) and/or a compound of formula (II), as compared to currently-available compositions. The compositions that include a high concentration of a compound of formula (I) and/or a compound of formula (II) provide a higher initial dose and a higher total dose, and can be administered orally or through inhalation. Initial administration of the compositions that include a high concentration of a compound of formula (I) and/or a compound of formula (II) act as a loading dose that quickly achieve therapeutic drug concentrations and prompt a rapid physiological response. The compositions disclosed herein that include a high concentration of a compound of formula (I) and/or a compound of formula (II) not only provide rapid onset, but also longer therapeutic duration than lower-dose compositions.

In some embodiments, the methods disclosed herein relate to administration of high-concentration a compound of formula (I) and/or a compound of formula (II) from a vaporizing device. In some embodiments, the methods disclosed herein relate to administration of high-concentration liquid compositions comprising a compound of formula (I) and/or a compound of formula (II). By employing high-concentration compositions comprising a compound of formula (I) and/or a compound of formula (II), the inventors developed a method that provides a higher total dose, as compared to currently-available 7-hydroxymitragynine and mitragynine pseudoindoxyl delivery methods. Compositions that comprise both a compound of formula (I) and a compound of formula (II) can include a compound of formula (I) to compound of formula (II) mass: mass ratio that is any one of, less than, greater than, or between 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, or any range derivable therein.

Methods of the present disclosure include but are not limited to methods of managing pain in a subject, methods for treating cough, methods for treating anxiety, methods for treating depression, methods for treating opioid use disorder, and methods for treating opioid withdrawal, wherein the methods comprise administering a composition as disclosed herein.

The methods disclosed herein can be adapted for use with a variety of other components having similar biophysical and/or chemical properties to 7-hydroxymitragynine, including but not limited to mitragynine, speciociliatine, paynantheine, corynantheidine, speciogynine, mitraphylline, rhynchophylline, mitralactonal, raubasine, tetrahydrocannabinol, including 4-8, 4-9, and A-10 tetrahydrocannabinol, hexahydrocannabinol, and tetrahydrocannabinol-A.

The compositions disclosed herein can be included in an edible product. Non-limiting examples of edible products include a lozenge, candy (including hard candies/boiled sweets, lollipop, gummy candy, candy bar, etc.), chocolates, brownie, cookie, trail bar, crackers, dissolving strip, mint, pastry, and bread.

The compositions disclosed herein can include a delivery-enhancing additive. Delivery-enhancing additives can be used to increase the total concentration of compounds of formula (I) and/or formula (II) that are delivered to a subject. Without being bound by theory, it is believed that delivery-enhancing additives can help volatilize formulations that include one or more compounds of formula (I) and/or formula (II) and a delivery-enhancing additive. In some aspects, a delivery-enhancing additive has a vapor pressure greater than that of 7-hydroxymitragynine. In some aspects, a delivery-enhancing additive has a vapor pressure greater than that of mitragynine pseudoindoxyl. In some embodiments, a solvent is employed as a delivery-enhancing additive. In some embodiments, a surfactant is employed as a delivery-enhancing additive. Surfactants useful as the surfactant components in the compositions of the present invention include nonionic, anionic, cationic, amphoteric, and zwitterionic surfactants, and may be used in combination with each other. Non-limiting examples of delivery-enhancing additives include ethanol, propylene glycol, glycerin, lecithin, cyclodextrin, water, kolliphor, castor oil, TPGS, soy lecithin, sunflower lecithin, polysorbate, propylene glycol, glycerin, glucose, cyclodextrins. The mass: mass ratio of a composition (that contains a compound of formula (I) and/or a compound of formula (II)) to delivery-enhancing additive may be any one of, less than, greater than, or between 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or any range derivable therein. It is also contemplated that certain delivery-enhancing additives, including one or more of the additives disclosed herein, can be excluded from compositions of the present invention.

In some embodiments, sources of 7-hydroxymitragynine include any chemical capable of providing a volatile form of 7-hydroxymitragynine, such as the free 7-hydroxymitragynine base or a 7-hydroxymitragynine salt (e.g. 7-hydroxymitragynine hydrochloride). In some embodiments, two sources of 7-hydroxymitragynine are used. For example, a composition as disclosed herein can include 7-hydroxymitragynine and a 7-hydroxymitragynine salt.

In some embodiments, sources of mitragynine pseudoindoxyl include any chemical capable of providing a volatile form of mitragynine pseudoindoxyl, such as the free mitragynine pseudoindoxyl base or a mitragynine pseudoindoxyl salt (e.g., mitragynine pseudoindoxyl hydrochloride). In some embodiments, two sources of mitragynine pseudoindoxyl are used. For example, a composition as disclosed herein can include mitragynine pseudoindoxyl and a mitragynine pseudoindoxyl salt.

In some embodiments, a composition as disclosed herein can include an antioxidant for stabilizing and/or reducing the degradation rate of the composition. Non-limiting examples of antioxidants that can be used with the compositions that comprise a compound of formula (I) and/or a compound of formula (II) include ascorbic acid, sodium ascorbate, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, tocopherol, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, dioleyl tocopheryl methylsilanol, cysteine, cysteine hydrochloride, diamylhydroquinone, di-t-butylhydroquinone, disodium ascorbyl sulfate, dodecyl gallate, erythorbic acid, thyl ferulate, ferulic acid, gallic acid esters, hydroquinone, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium bisulfite, sodium erythorbate, sodium metabisulfite, sodium sulfite, superoxide dismutase, sorbityl furfural, and tris (nonylphenyl) phosphite. It is also contemplated that certain antioxidants, including one or more of the antioxidants disclosed herein, can be excluded from compositions of the present invention.

In some embodiments, a composition as disclosed herein can include a preservative. Non-limiting examples of preservatives that can be used include parabens (e.g., methylparabens and propylparabens), phenoxyethanol, benzyl alcohol, chlorobutanol, phenol, sorbic acid, thimerosal, and quaternary ammonium preservatives such as polyquaternium-1 and benzalkonium halides. It is also contemplated that certain preservatives, including one or more of the preservatives disclosed herein, can be excluded from compositions of the present invention.

In some embodiments, a composition as disclosed herein includes a flavoring agent. As used herein, a “flavoring agent” is any flavorful or aromatic substance capable of altering the sensory characteristics associated with the compositions comprising a compound of formula (I) and/or a compound of formula (II) disclosed herein. Examples of sensory characteristics that can be modified by the flavoring agent include taste, mouthfeel, moistness, coolness/heat, and/or fragrance/aroma. Non-limiting examples of flavoring agents include vanillin, vanilla extract, mango extract, cinnamon, citrus, coconut, ginger, viridiflorol, almond, menthol, grape skin extract, and grape Seed extracts include, but are not limited to. A flavoring agent can include natural or synthetic materials, or a combination thereof. Flavoring agents also include any other materials that impart flavor, and include any natural or synthetic materials that are generally recognized as safe for human consumption. It is also contemplated that certain flavoring agents, including one or more of the flavoring agents disclosed herein, can be excluded from compositions of the present invention.

In some embodiments, a composition as disclosed herein can include a tobacco material. The tobacco material can vary in species, type, and form. Tobacco materials are generally obtained from for a harvested plant of the Nicotiana species. In some embodiments, the tobacco material is an extract or an oil of a plant of the Nicotiana species. Non-limiting examples of Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. It is also contemplated that certain tobacco materials, including one or more of the tobacco materials disclosed herein, can be excluded from compositions of the present invention.

In embodiments where the composition is in a liquid form, a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof can be included in the composition. The moisture content of the composition, prior to use by a consumer, can vary according to desired properties. In some embodiments, the moisture content is at least partially related to the content of other components within the composition, such as the content of a compound of formula (I), the content of a compound of formula (I), or content of another component within the composition. It is also contemplated that water can be excluded from compositions of the present invention.

In some embodiments, a composition as disclosed herein includes one or more further additives. The compositions disclosed herein can be processed, blended, formulated, combined and/or mixed with other materials or ingredients. The additives can be natural or synthetic. It is also contemplated that additives, including one or more of the additives disclosed herein, can be excluded from compositions of the present invention.

The compositions of the present invention can be administered orally, via inhalation (e.g., aerosol inhalation), intravenously, intradermally, intraarterially, intraperitoneally, intralesionally, intracranially, intraarticularly, intraprostaticaly, intrapleurally, intratrachcally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, locally, via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990). The actual dosage amount of a composition of the present invention administered to a subject can be determined by physical and physiological factors such as body weight, severity of condition being treated, previous or concurrent therapeutic interventions, idiopathy of the patient and on the route of administration.

The compound of formula (I) and/or compound of formula (II) may be formulated into a composition in a free base, neutral, or salt form. Suitable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In some embodiments, methods for administering a compound of formula (I) and/or a compound of formula (II) to a subject involve a step of increasing the temperature of a composition as disclosed herein. Such temperature-increasing steps are generally used to volatilize components within the composition. In some embodiments, temperature-increasing steps are used to regulate the rate of delivery of a compound of formula (I) and/or a compound of formula (II). In some embodiments, a temperature gradient is employed, where the temperature increase is provided over a period of time. In some embodiments, a lower temperature increase is used first to warm a composition as disclosed herein. This can be performed to reduce the viscosity of a composition or to help liquify a composition, for example. In some embodiments, the temperature is then increased over time to volatilize components within the composition. In other embodiments a constant temperature is maintained during use. In some embodiments, the temperature is increased to a temperature that is any one of, less than, greater than, or between 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, and 315° C.

In some embodiments, the methods described herein are carried out using a device configured to carry out the methods described herein during device operation. The compositions disclosed herein can be contained within a device, then the device can be used to administer a therapeutically effective dose of a compound of formula (I) and/or a compound of formula (II). In some embodiments, the device is a vaporizing device or an electronic cigarette device. In some embodiments, the vaporizing or electronic cigarette device includes a heating element and/or a temperature control. The vaporizing or electronic cigarette device heating element and/or temperature control can be adjusted to monitor the rate of delivery of a compound of formula (I) and/or a compound of formula (II). In some embodiments, the vaporizing or electronic cigarette device is a metered dose device that can administer a predetermined amount of a compound of formula (I) and/or a compound of formula (II). In some embodiments, the device is an aerosolizing device that employs a propellant gas to propel a liquid or vaporized composition comprising a compound of formula (I) and/or a compound of formula (II). In some embodiments, the aerosolizing device is a metered dose device that can administer a predetermined amount of a composition that comprises a compound of formula (I) and/or a compound of formula (II). In some embodiments, the aerosolizing device is a nebulizer device.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” includes straight-chain alkyl, branched-chain alkyl, cycloalkyl (alicyclic), heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃) CH₂CH₃ (sec-butyl), —CH₂CH(CH₃)₂ (iso-butyl), —C(CH₃)₃ (tent-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, piperidinyl, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH2CH2OC(O)CH3, —CH₂CH₂NHCO₂C(CH₃)₃, and —CH₂Si(CH₃)₃.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, C₆H₄CH(CH₂)₂, —C₆H₃(CH₃) CH₂CH₃, C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH═, —C₆H₄CF₃, —C₆H₄CN, —C₆H₄CHO, —C₆H₄CHO, —C₆H₄C (O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents. The term “halide” includes —F, —Cl, —Br, and —I.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Mitragynine (1000 g, 2.5 mol, 1 equiv.) was dissolved in a mixture of MeCN: H₂O and added to a 100 L reactor. The reactor was chilled to 0° C. Phenyliodine bis(trifluoroacetate) (PIFA, 1182.61 g, 2.75 mol, 1.1 equiv.) was added in one batch and conversion of mitragynine to 7-hydroxymitragynine was tracked using HPLC. 7-hydroxmitragynine is converted to mitragynine pseudoindoxyl using zinc triflate following literature procedure¹¹ and is purified in a similar manner to 7-hydroxymitragynine.

### Example 2

7-hydroxymitragynine was purified using column chromatography. 7 kg to 10 kg batches are dissolved in a polar aprotic solvent and run on a 45 kg silica column. Fractions of interest are collected, combined after HPLC confirmation, and concentrated in vacuo.

### Example 3

### Tail Flick Test Overview

The objective of the experiments described below was to evaluate and compare the analgesic effects of 7-hydroxymitragynine administered orally and via inhalation. Ten (10) wildbred mice (5 male and 5 female) were purchased and each placed into a small plastic tote with bedding designed to house an individual mouse. Average mouse mass was 32 g. Oral administration doses of 7-hydroxymitragynine ranged from 1.5-11 μg, and inhalation doses ranged from 4-30 μg.

Tail flick tests were performed on the mice in order to examine analgesic effects of 7-hydroxymitragynine. A baseline temperature of 25° C. was used as the control and temperatures of 30° C., 40° C. and 45° C. were used for pain threshold analyses. Each mouse was restrained while the distal half of its tail is immersed in a water bath at the indicated temperatures, and the time required for the mouse to flick (i.e., withdraw) its tail from the water bath stimulus was recorded. A flick outside of the water was determined to be a tail flick away from the water.⁸⁻¹⁰ The number of tail flicks within a 30-second period was measured.

### Example 4

### Oral Administration Study

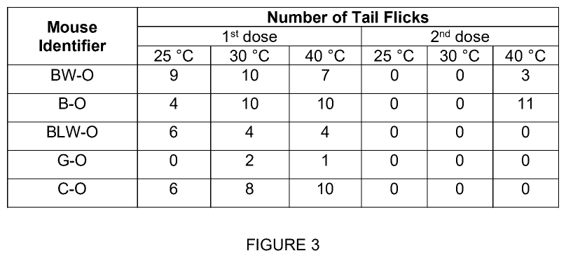

Control mice and mice administered 7-hydroxymitragynine orally (oral test mice) were subjected to tail flick analyses at the indicated temperatures (FIG. 3). Oral test mice were tested 30 min after oral administration of a first dose of 7-hydroxymitragyninc.

At 25° C., the average number of tail flicks for control mice was 6.5 tail flicks. Test mouse G-O did not show any visible tail flicks. Test mice BLW-O and C-O presented 6 tail flicks.

At 30° C., the average number of tail flicks for control mice was 10 tail flicks. Test mouse G-O presented 2 tail flicks. Test mouse BLW-O presented 4 tail flicks. Test mouse C-O presented 8 tail flicks.

At 40° C., the average number of tail flicks for control mice was 8.5 tail flicks. Test mouse BLW-O presented 4 tail flicks. Test mouse G-O presented 1 tail flick. Test mouse C-O presented 10 test flicks, matching the no-dose control.

30 minutes after the first round of tests discussed above, a second dose of 7-hydroxymitragynine was administered orally, and mice were re-tested after a wait time of 1 hour. At 40° C., the average number of tail flicks for control mice was 7 tail flicks. Test mouse G-O presented 1 tail flick. Test mice BLW-O presented 3 tail flicks. Test mouse C-O presented 3 tail flicks. As seen in FIG. 3, oral test mice showed a marked reduction in the number of tail flicks after the second dose of 7-hydroxymitragynine. The reduction in tail flick number is indicative of the analgesic effects imparted by 7-hydroxymitragyninc.

### Example 5

### Behavioral Characteristics-Oral Administration

The oral administration study control mice exhibited common demeanor of mice that have general well-being. The mice did exhibit stressed behavior when placed into the tail flick test vessel. When removed from the vessel, the mice calmed down and exhibited general demeanor.

Upon administration of the first dose of 7-hydroxymitragynine, oral test mice exhibited no changes in demeanor. Oral test did exhibit stressed behavior when placed into the tail flick test vessel. The stressed behavior diminished when removed from the test vessel.

Slower behavior was observed 30 minutes after 7-hydroxymitragynine administration, and prior to initiation of tail flick testing. The highest and median dose mice, BLW and G, respectively, exhibited had no changes in coordination or respiratory rate, but exhibited lethargic behavior.

Upon administration of the second dose of 7-hydroxymitragynine, oral test mice had slack facial features, and were much calmer exhibited increased lethargy. Respiration rate did not appear to change. 75 minutes after the second dose, oral test mice were less active than control counterparts, but did not exhibit negative side effects. The decrease in tail flicks at high temperatures indicated a dose-dependent increase in pain tolerance.

### Example 6

### Inhalation Administration Study

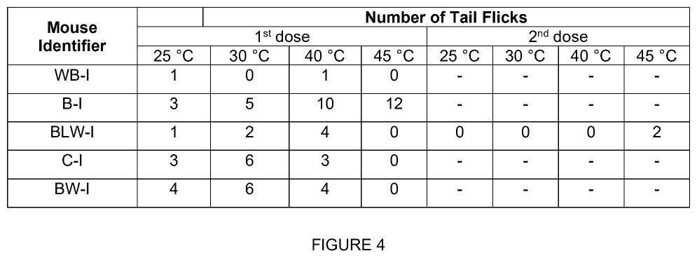

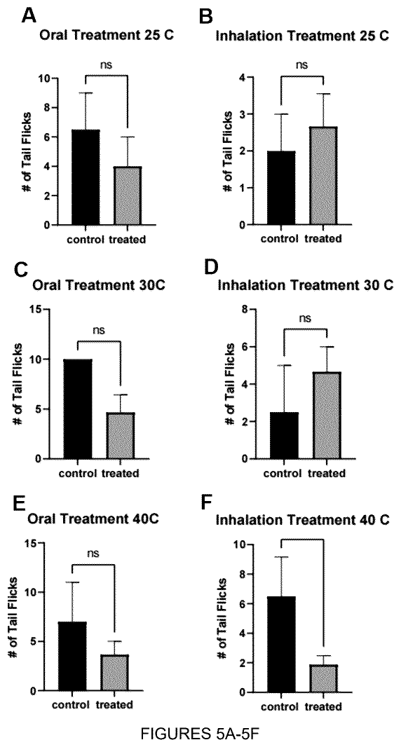

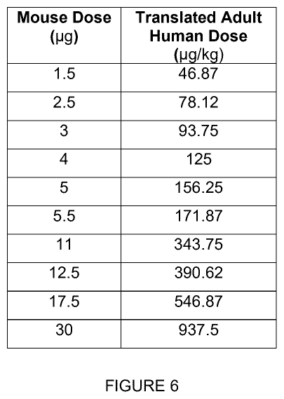

Control mice and mice administered 7-hydroxymitragynine via inhalation (inhalation test mice) and were subjected to tail flick analyses at the indicated temperatures (FIG. 4). Oral test mice were tested 30 min after inhaled administration of a first dose of 7-hydroxymitragynine. The 7-hydroxymitragynine was provided at a concentration of 80%.

At 25° C., the average number of tail flicks for control mice was 2 tail flicks. Test mouse G did not show any visible tail flicks. Test mouse BLW-I presented 1 tail flick. Test mouse C-I presented 3 tail flicks. Test mouse BW-I presented 4 tail flicks.

At 30° C., the average number of tail flicks for control mice was 5 tail flicks. Test mouse BLW-I presented 2 tail flicks. Test mice C-I and BW-I presented 6 tail flicks.

At 40° C., the average number of tail flicks for control mice was 5.5 tail flicks. Test mouse BLW-I presented 4 tail flicks. Test mouse C-I presented 3 tail flicks. Test mouse BW-I presented 4 tail flicks.

Immediately after the first round of tests discussed above, a second dose of 7-hydroxymitragynine was administered via inhalation (total of 1^{st} and 2^{nd} doses=30 μg), and mice were re-tested immediately at 45° C. One control mouse was removed from the test due to unreliable response (outlier). The average number of tail flicks for the remaining control mouse was 12 tail flicks. Test mouse BLW-I presented 2 tail flicks. As seen in FIG. 4, inhalation test mouse showed a marked reduction in the number of tail flicks after the second dose of 7-hydroxymitragynine. The reduction in tail flick number is indicative of the analgesic effects imparted by 7-hydroxymitragynine.

### Example 7

### Behavioral Characteristics—Inhalation Administration

The inhalation administration study control mice exhibited a general well-being demeanor, with no signs of stress or sickness prior to the tail flick assessment. The mice did exhibit stressed behavior when placed into the tail flick test vessel. When removed from the vessel, the mice calmed down and exhibited general demeanor.

Inhalation test mice did not exhibit stressed behavior prior to administration of 7-hydroxymitragynine. During inhalation administration of the first dose, mouse BW-I's tail was injured. Its behavior was monitored closely, and it did not respond in the usual manner. Instead, mouse BW-I was calm after the first dose, and tended to grooming its tail and eating food. Mouse BW-I did not exhibit stressed behavior or pain throughout the assessment, indicating effectual analgesia imparted by the inhaled dose.

The remaining inhalation test also demonstrated a reduction in the number of tail flicks at higher water temperatures, confirming the analgesic effects of inhaled 7-hydroxymitragynine. Test mouse C-I received the second half of a full 30 microgram dose and did exhibit slow movement, and calm behavior. Its demeanor was not stressed.

All inhalation test mice responded calmly to stimuli such as being picked up and handled. Movements were slower, indicating almost immediate effects of inhaled 7-hydroxymitragynine.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Todd, D. A.; Kellogg, J. J.; Wallace, E. D.; Khin, M.; Flores-Bocanegra, L.; Tanna, R. S.; McIntosh, S.; Raja, H. A.; Graf, T. N.; Hemby, S. E.; Paine, M. F.; Oberlies, N. H.; Cech, N. B. Chemical Composition and Biological Effects of Kratom (Mitragyna speciosa): In Vitro Studies with Implications for Efficacy and Drug Interactions. Sci. Rep. 2020, 10 (1), 1-13.
2. Hemby, S. E.; McIntosh, S.; Leon, F.; Cutler, S. J.; McCurdy, C. R. Abuse Liability and Therapeutic Potential of the Mitragyna speciosa (Kratom) Alkaloids Mitragynine and 7-hydroxymitragynine. Addict. Biol. 2019, 24 (5), 874-885.
3. Suhaimi, F. W.; Yusoff, N. H. M.; Hassan, R.; Mansor, S. M.; Navaratnam, V.; Müller, C. P.; Hassan, Z. Neurobiology of Kratom and Its Main Alkaloid Mitragynine. Brain Res. Bull. 2016, 126, 29-40.
4. Kratom (Mitragyna speciosa) drug profile. Europa.eu. https://www.emcdda.europa.cu/publications/drug-profiles/kratom_en (accessed 2024 Jun. 25).
5. Zhang, M.; Sharma, A.; León, F.; Avery, B.; Kjelgren, R.; McCurdy, C. R.; Pearson, B. J. Plant Growth and Phytoactive Alkaloid Synthesis in Kratom [Mitragyna speciosa (Korth.)] in Response to Varying Radiance. PLOS One 2022, 17 (4), c0259326.
6. Manda, V.; Avula, B.; Ali, Z.; Khan, I.; Walker, L.; Khan, S. Evaluation of in Vitro Absorption, Distribution, Metabolism, and Excretion (ADME) Properties of Mitragynine, 7-Hydroxymitragynine, and Mitraphylline. Planta Med. 2014, 80 (07), 568-576.
7. Ponglux, D.; Wongseripipatana, S.; Takayama, H.; Kikuchi, M.; Kurihara, M.; Kitajima, M.; Aimi, N.; Sakai, S.-I. A New Indole Alkaloid, 7 α-Hydroxy-7H-Mitragynine, from Mitragyna Speciosain Thailand. Planta Med. 1994, 60 (06), 580-581.
8. Guarnieri, M.; Schildhaus, N.; Trink, E.; Polson, C.; DeTolla, L.; Tyler, B.; Jallo, G.; Tok, S. Thermal Latency Studies in Opiate-Treated Mice. J. Pharm. Bioallied Sci. 2014, 6 (1), 43.
9. Hole, K.; Tjølsen, A. Tail Flick Test. In Encyclopedia of Pain; Springer Berlin Heidelberg: Berlin, Heidelberg, 2006; pp 2392-2395.
10. Bannon, A. W.; Malmberg, A. B. Models of Nociception: Hot-plate, Tail-flick, and Formalin Tests in Rodents. Curr. Protoc. Neurosci. 2007, 41 (1).
11. Váradi A, et al. Mitragynine/corynantheidine pseudoindoxyls as opioid analgesics with Mu agonism and delta antagonism, which do not recruit β-arrestin-2. J. Med. Chem. 2016; 59:8381-8397.

1. (canceled)

2. The vaporizing device of claim 28, wherein the composition comprises from 10% to 99% by weight of the compound of formula (I), based on a total weight of the composition.

3. The vaporizing device of claim 28, wherein the composition comprises from 10% to 99% by weight of the compound of formula (II), based on a total weight of the composition.

4. The vaporizing device of claim 28, wherein the composition comprises a solvent in an amount ranging from 1% to 90% by weight, based on a total weight of the composition.

5. (canceled)

6. (canceled)

7. The vaporizing device of claim 28, wherein the composition further comprises at least one of an antioxidant, a preservative, a flavoring agent, a surfactant, and a tobacco material.

8. A method for administering a composition comprising a compound of formula (I) and/or a compound of formula (II), to a subject, the method comprising obtaining a composition comprising at least 10% of a compound of formula (I) by weight, at least 10% of a compound of formula (II) by weight, or at least 10% by weight of a combination of a compound of formula (I) and a compound of formula (II), based on a total weight of the composition, 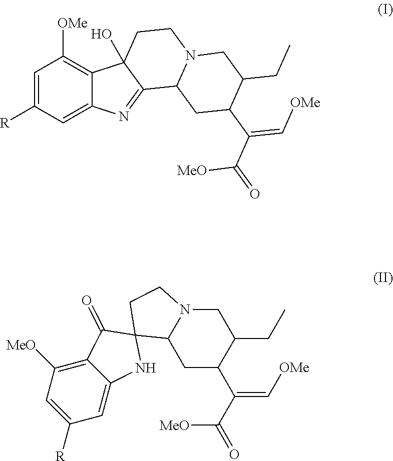
where R is H, halide, C₁-C₆ alkyl, aryl, nitrile or —CONH₂; and
administering the composition to the subject through use of a vaporizing device.

9. The method of claim 8, wherein the composition comprises from 10% to 99% by weight of the compound of formula (I), based on the total weight of the composition.

10. The method of claim 8, wherein the composition comprises from 10% to 99% by weight of the compound of formula (II), based on the total weight of the composition.

11. The method of claim 8, wherein the composition is formulated as an oil, solution, suspension, tablet, capsule, lozenge, candy, gummy, chocolate, brownie, cookie, trail bar, cracker, dissolving strip, or mint.

12. The method of claim 8, wherein the composition is formulated as a liquid.

13. The method of claim 8, wherein the composition comprises a solvent in an amount ranging from 1% to 90% by weight, based on a total weight of the composition.

14. The method of claim 8, wherein the compound of formula (I) is 7-hydroxymitragynine.

15. The method of claim 8, wherein the compound of formula (II) is mitragynine pseudoindoxyl.

16. The method of claim 8, wherein the composition further comprises at least one of an antioxidant, a preservative, a flavoring agent, a surfactant, and a tobacco material.

17. The method of claim 8, wherein the method further comprises volatilizing the composition and inhaling the volatilized composition.

18. A method for inducing an analgesic effect in a subject,
the method comprising obtaining a composition comprising at least 10% of a compound of formula (I) by weight, at least 10% of a compound of formula (II) by weight, or at least 10% by weight of a combination of a compound of formula (I) and a compound of formula (II), based on a total weight of the composition, and administering the composition to the subject through use of a vaporizing device, 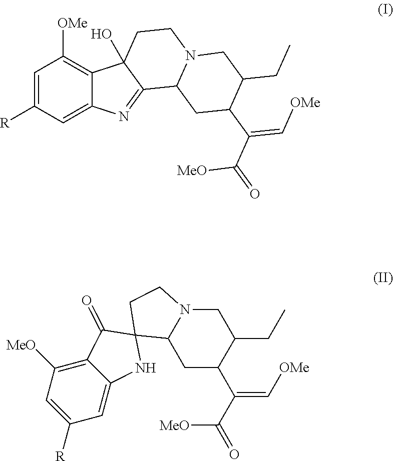
where R is H, halide, C₁-C₆ alkyl, aryl, nitrile or —CONH₂; and
wherein administration of the composition leads to an analgesic effect in the subject.

19. The method of claim 18, wherein the composition comprises from 10% to 99% by weight of the compound of formula (I), based on the total weight of the composition.

20. The method of claim 18, wherein the composition comprises from 10% to 99% by weight of the compound of formula (II), based on the total weight of the composition.

21. The method of claim 18, wherein the composition is formulated as an oil, solution, suspension, tablet, capsule, lozenge, candy, gummy, chocolate, brownie, cookie, trail bar, cracker, dissolving strip, or mint.

22. The method of claim 18, wherein the composition is formulated as a liquid.

23. The method of claim 18, wherein the composition comprises a solvent in an amount ranging from 1% to 90% by weight, based on a total weight of the composition.

24. The method of claim 18, wherein the composition further comprises at least one of an antioxidant, a preservative, a flavoring agent, and a tobacco material.

25. The method of claim 18, wherein the method further comprises volatilizing the composition; and inhaling the volatilized composition.

26. The method of claim 18, wherein the compound of formula (I) is 7-hydroxymitragynine.

27. The method of claim 18, wherein the compound of formula (II) is mitragynine pseudoindoxyl.

28. A vaporizing device comprising a composition that includes at least 10% of a compound of formula (I) by weight, at least 10% of a compound of formula (II) by weight, or at least 10% by weight of a combination of a compound of formula (I) and a compound of formula (II), based on a total weight of the composition; 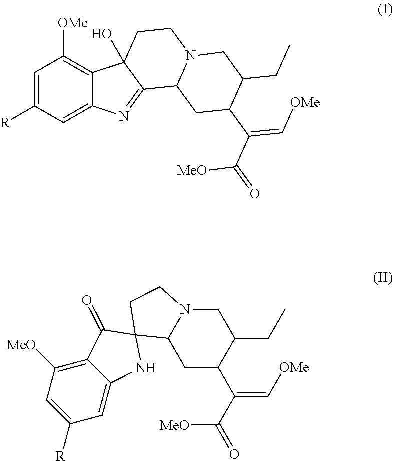
where R is H, halide, C₁-C₆ alkyl, aryl, nitrile or —CONH₂.

29. The vaporizing device of claim 28, wherein the compound of formula (I) is 7-hydroxymitragynine.

30. The vaporizing device of claim 28, wherein the compound of formula (II) is mitragynine pseudoindoxyl.
